(19) United States
(12) Patent Application Publication
(10) Pub. No.: US 2026/0031191 A1
(43) Pub. Date: Jan. 29, 2026

(54) METHOD FOR INTEGRATION OF SORPTION MATERIALS AND DATA ANALYSIS FOR DETECTION OF CONTAMINANTS IN FLUIDS

(71) Applicant: Max-IR Labs, Incorporated, Dallas, TX (US)

(72) Inventors: Ecatherina Roodenko Fuchs, Plano, TX (US); Trey Daunis, Plano, TX (US)

(73) Assignee: Max-IR Labs, Incorporated, Dallas, TX (US)

(21) Appl. No.: 19/277,335

(22) Filed: Jul. 22, 2025

Related U.S. Application Data
(60) Provisional application No. 63/674,551, filed on Jul. 23, 2024.

(51) Int. Cl. G16C 20/10; G01N 21/31; G01N 27/02

(52) U.S. Cl. CPC G16C 20/10; G01N 21/31; G01N 27/02

(57) ABSTRACT

A method for detection of an analyte in a fluid. The method includes i) trapping the analyte in the fluid using a sorption material; ii) continuously measuring time-dependent accumulation data of the trapped analyte in the sorption material using a measurement apparatus over a plurality of time points prior to, or including, equilibrium; iii) fitting the time-dependent accumulation data to a kinetic model during the measurement process; and iv) predicting an equilibrium concentration value of the analyte in the fluid based on the fitted kinetic model at any time during the sorption process, including prior to or at equilibrium

### CROSS-REFERENCE TO RELATED APPLICATION(S) AND CLAIM OF PRIORITY

The present application is related to U.S. Provisional Patent No. 63/674,551, filed Jul. 23, 2024, entitled “Method For Integration Of Sorption Materials And Data Analysis For Detection Of Contaminants In Fluids”. Provisional Patent No. 63/674,551 is assigned to the assignee of the present application and is hereby incorporated by reference into the present application as if fully set forth herein. The present application hereby claims priority under 35 U.S.C. § 119(e) to U.S. Provisional Patent No. 63/674,551.

### TECHNICAL FIELD

The present application relates generally to a method for detection of contaminants in fluids.

### BACKGROUND

Analysis of biological microorganisms and other contaminants in fluids is of interest in healthcare, the pharmaceutical industry, the food and beverage industry, the routine screening of drinking water, and many other fields. Detection of different chemicals in fluids is a complex task that requires one to distinguish between the fluid matrix (e.g., water, ethanol, oil) and the analytes of interest (e.g., ammonia in water, methanol in ethanol, sulfur in petroleum). The presence of interfering substances in fluids can obscure the target analytes and reduce detection sensitivity and specificity. Achieving inline, low-concentration detection with high accuracy and selectivity in diverse and often harsh environmental conditions further complicates the task.

Therefore, there is a need for methods and apparatuses for accurately detecting low concentrations of analyte chemicals in fluids.

### SUMMARY

To address the above-discussed deficiencies of the prior art, it is a primary object to provide a method for detection of an analyte in a fluid. The method includes i) trapping the analyte in the fluid using a sorption material; ii) continuously measuring time-dependent accumulation data of the trapped analyte in the sorption material using a measurement apparatus over a plurality of time points prior to, or including, equilibrium; iii) fitting the time-dependent accumulation data to a kinetic model during the measurement process; and iv) predicting an equilibrium concentration value of the analyte in the fluid based on the fitted kinetic model at any time during the sorption process, including prior to or at equilibrium.

In an embodiment of the disclosure, the kinetic model describes the rate of analyte sorption and comprises one or more kinetic models selected from mechanistic, empirical, or rate-based models, including but not limited to Langmuir adsorption kinetics, pseudo-first-order, pseudo-second-order, intraparticle diffusion, Elovich-type models, or data-driven predictive models, including but not limited to: regression, curve-fitting, or machine-learning-based models trained to infer equilibrium behavior based on sequential measurements; and wherein the kinetic model comprises a pseudo-second-order kinetic expression qₜ/dt=k₂(qₜ−qₑ)², where: qₜ is the amount of analyte in the sorption material at time t, k₂ is the pseudo-second-order rate constant, and qₑ is the equilibrium concentration value of the analyte in the sorption material.

In another embodiment, the measuring apparatus applies the kinetic model to the time-dependent accumulation data in real time during the measurement process to dynamically estimate the equilibrium concentration of the analyte in the sorption material and infer the concentration of the analyte in fluid.

In still another embodiment, the measurement apparatus determines the time dependent accumulation data by analyzing a time-dependent signal.

In yet another embodiment, the time-dependent signal comprises an optical signal, including spectroscopic signals.

In a further embodiment, the measurement apparatus predicts the equilibrium concentration value by fitting a kinetic profile of the time-dependent optical signal, including spectroscopic data, collected during the sorption process.

In a still further embodiment, the time-dependent signal comprises an electrical signal.

In a yet further embodiment, the measurement apparatus predicts the equilibrium concentration value by fitting a kinetic profile of the time-dependent electrical signal collected during a process of trapping the analyte.

In an embodiment, the kinetic model fitting is dynamically updated in real time with each subsequent measurement point collected during the process of trapping the analyte, thereby continuously refining the predicted equilibrium concentration value of the analyte.

In another embodiment, the accuracy of the predicted equilibrium concentration value improves progressively as additional time-dependent measurement data points are collected and incorporated into the kinetic model fitting.

In still another embodiment, the fluid comprises one of i) water; ii) an organic or inorganic solvent; or iii) a non-aqueous fluid.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates an optical instrument which integrates sorption material according to an embodiment of the disclosure.

FIG. 2 illustrates electrical instrument which integrates sorption material using an electric signal (e.g., impedance, resistance, current, voltage, etc.) for sensing an analyte according to an embodiment of the disclosure.

FIG. 3 illustrates an optical instrument which integrates sorption material using infrared spectral range radiation for sensing an analyte according to an embodiment of the disclosure.

FIG. 4 illustrates an ion-exchange resin according to an embodiment of the disclosure.

FIG. 5 illustrates an ion-exchanged ionomer according to an embodiment of the disclosure,

FIG. 6 illustrates activated carbon 600 according to an embodiment of the disclosure.

FIG. 7 is a graph illustrating an IR peak area of the C—F bond showing development as a function of time, during PFAS trapping in FC waveguide coating according to an embodiment of the disclosure.

FIG. 8 is a graph illustrating calculated IR peak area of PFAS as a function of time in an FC coated waveguide according to an embodiment of the disclosure.

FIG. 9 is a graph illustrating calculated IR peak area of PFAS as a function of time in an NP coated waveguide according to an embodiment of the disclosure.

FIG. 10 is a graph illustrating IR peak area during monitoring of FC coating regeneration salt solution according to an embodiment of the disclosure.

FIG. 11 is a graph illustrating IR absorption peaks of 1 ppm, 8 ppm, and 16 ppm nitrate solutions measured using an uncoated fiber according to an embodiment of the disclosure.

FIG. 12 is a graph illustrating IR absorption peaks of 1 ppm nitrate solution measured using a coated fiber according to an embodiment of the disclosure.

FIG. 13 is a graph illustrating nitrate IR absorption peak area from measurements shown in FIG. 12 plotted as a function of time according to an embodiment of the disclosure.

FIG. 14A is a graph that shows data linearized according to a pseudo-second-order kinetic model with a fit line according to an embodiment of the disclosure.

FIG. 14B is a graph that shows the original data of peak area vs. time axes with pseudo-second-order fit line according to an embodiment of the disclosure

FIG. 15A is a graph that shows the predictive equilibrium peak area (left y-axis) vs. time, with percent variation from the final value (right y-axis) according to an embodiment of the disclosure.

FIG. 15B is a graph that shows the predictive peak area vs. time as in FIG. 15a with a 10 data point moving average window applied according to an embodiment of the disclosure.

FIG. 16 is a graph that shows the IR absorption spectrum from the C—F bonds of a 25 ppm solution of PFOA in water using an uncoated fiber according to an embodiment of the disclosure.

FIG. 17 is a graph that shows the IR absorption spectrum of a 25 ppm solution of PFOA in water measured using a coated fiber according to an embodiment of the disclosure.

FIG. 18 is a graph that shows the PFOA IR absorption peak area from measurements shown in FIG. 17 plotted as a function of time according to an embodiment of the disclosure.

FIG. 19A is a graph that shows data linearized according to a pseudo-second-order kinetic model (circles) with a fit line according to an embodiment of the disclosure.

FIG. 19B is a graph that shows the original data (circles) on peak area vs. time axes with pseudo-second-order fit line from FIG. 19a according to an embodiment of the disclosure.

FIG. 20A is a graph illustrating predictive equilibrium peak area (left y-axis) vs. time with percent variation from the final value (right y-axis) according to an embodiment of the disclosure.

FIG. 20B is a graph illustrating the predictive peak area vs. time as in FIG. 20a with a 10 data point moving average window applied according to an embodiment of the disclosure.

FIG. 21 illustrates a method for detection of an analyte in a fluid according to an embodiment of the disclosure.

### DETAILED DESCRIPTION

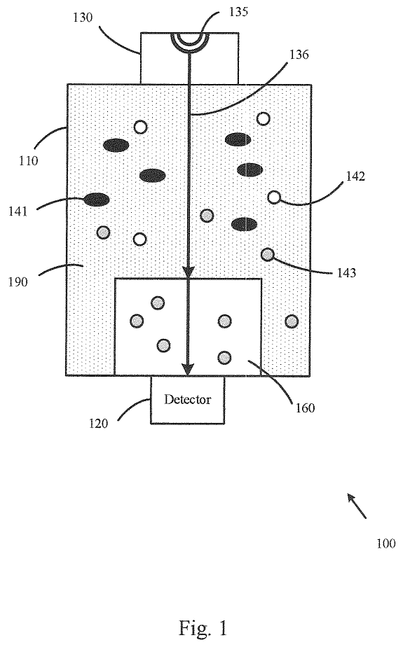

FIGS. 1 through 21, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged biofluid monitoring apparatus.

The present disclosure describes a “trap and measure” sensing method that enables monitoring of chemicals in fluids. As used herein, fluids may include water, organic or inorganic solvents, and other non-aqueous fluids (e.g., ethanol, oils, benzene). Fluids may also be referred to as “matrix” or “host fluids”. Analysis of contaminants in fluids is helpful for estimation of the purity of fluids and control of various industrial processes. The analyzed chemicals that can serve as contaminants of these fluids are listed below.

The disclosed method includes a physical medium (i.e., trapping or sorption material) that can be integrated in various measurement apparatuses and analytical tools. The disclosed method also includes a data analysis methodology for quantitative assessment of analytes in fluids. The measurement apparatus in which the physical medium (i.e., sorption material) is integrated may be an optical, spectroscopic or electronic apparatus that can quantitatively measure and monitor accumulation of a trapped analyte. Hereinafter, the “physical medium” may be referred to as “sorption material”. Examples of measurement apparatuses that integrate the sorption material are described below.

The disclosed method analyzes time-resolved signals—whether optical, electrical, or other measurable outputs—arising from analyte sorption onto functional materials, One important advantage of the method lies in using the kinetic profile of the signal to quantify contaminant concentration, regardless of the specific transduction mechanism, or specific sorbent. In this context, “transduction” refers to the process by which the analyte-sorption event is converted into a measurable signal—such as a change in optical absorption, electrical conductivity, or other physical property—that can be monitored over time. “Kinetic modeling” refers to the application of mathematical models to describe the rate and progression of analyte accumulation within the sorption material over time, enabling prediction of equilibrium behavior and contaminant concentration from early-time data.

Applications of the disclosed sensing method include remediation and industrial process control, including wastewater treatment, bioreactors, fermentation, environmental applications, food and beverage analysis, purity of pharmaceutical fluids, and others. The present disclosure describes a data analysis method that generally allows enhanced limits of detection (LOD) to detect analytes in fluids at concentrations below 0.1 ppt (parts per trillion) to over 10,000 parts per million (ppm).

The type of monitored chemicals (i.e., analytes) may include: i) nutrients such as nitrate, ammonia, phosphates, orthophosphates, potassium, magnesium, calcium, and the like; ii) inorganic carbon, including carbon dioxide (CO₂), bicarbonate (HCO₃⁻), carbonate (CO₃²⁻), carbonic acid (H₂CO₃), and other forms of inorganic carbon; iii) organic compounds and organic polymers, such as ethanol, methanol, methane, acetaldehyde, carboxylic acid, and the like; iv) amides (e.g., urea, nicotinamide); v) perfluoroalkyl substances (PFAS); vi) lipids, peptides, proteins, and the like; vii) sugars, monosaccharides and polysaccharides, and the like; viii) water (H₂O) (i.e., the amount of water can be detected in non-aqueous solutions); ix) metal and metal complexes (e.g., iron, lead, etc.).

The present disclosure is based on prior patents and applications assigned to Max-IR Labs, Incorporated and involves use of sorption materials for a pre-concentration in a “trap-and-measure” approach. The following sections describe the integration of the sorption materials with different measurements techniques, and data analysis. The prior patents include:
i) U.S. Pat. No. 11,344,883, entitled “Microfluidic Device With Integrated Waveguides For Analyte Identification And Method Of Operation Thereof:
ii) U.S. Pat. No. 10,613,025, entitled “Infrared Sensor For Soil Or Water And Method Of Operation Thereof”;
iii) U.S. Pat. No. 10,890,525, entitled “Infrared Analytical Sensor For Soil Or Water And Method Of Operation Thereof”;
iv) U.S. Pat. No. 10,458,907, entitled “Infrared Analytical Sensor For Soil Or Water And Method Of Operation Thereof”;
v) U.S. Pat. No. 10,883,930, entitled “Infrared Sensor For Soil Or Water And Method Of Operation Thereof”;
vi) U.S. Pat. No. 11,874,222 entitled “System And Method Using IR Radiation And Fluid Channel To Detect Contaminants And Salts In Fluids”.

U.S. Pat. Nos. 11,344,883, 10,613,025, 10,890,525, 10,458,907, 10,883,930, and 11,874,222 are hereby incorporated by reference in their entireties into the present application as if fully set forth herein.

Integration of sorption materials with different analytical tools for increased sensitivity and selectivity—Conventional optical instruments for fluid analysis operate by passing a radiation beam from a source through the fluid to a detector. As the beam passes through, it interacts with contaminants (analytes) in the fluid. When the absorption bands of the analyte match the radiation wavelength, the analyte absorbs the radiation. The absorption strength is directly correlated with the analyte concentration. However, if the analyte concentration is very low, the interaction may be negligible and make detection difficult. The “trap-and-measure” technique disclosed herein improves the sensitivity of the measurement. In addition to enhancing sensitivity through pre-concentration, the disclosed approach enables kinetic analysis of analyte sorption onto the functional material, allowing quantification of analyte concentration based on time-resolved signal dynamics rather than relying solely on equilibrium absorption strength.

FIG. 1 illustrates optical instrument 100 which integrates sorption material 160 according to an embodiment of the disclosure. Optical instrument 100 includes reservoir 110 that contains a liquid, such as water 190. Water 190 contains a plurality of analytes, including analytes 141 (black ovals), analytes 142 (white circles), and preferred analytes 143 (gray circles). Radiation source 130 includes optics 135 that generate probing beam 136 that is transmitted to detector 120. When sorption material 160 is placed in the path of probing beam 136, sorption material 160 traps and pre-concentrates preferentially trapped analyte 143. As beam 136 passes through sorption material 160, beam 136 interacts with a larger amount of trapped analyte 143, thereby enhancing the detection capability for the analyte of interest. Quantitative analysis can then be performed to estimate the amount of analyte 143 collected in sorption material 160.

Optical instrument 100 may operate in different spectral ranges, such as the visible (VIS), ultraviolet (UV), terahertz (THz), or infrared (IR) spectral ranges. The radiation from source 130 passes through sorption material 160 submersed in liquid (e.g., water) 190, with the radiation being absorbed at frequencies that are typical for the absorption bands of the analytes 143 of interest. For example, nitrate (NO₃) absorbs UV radiation in the range between 190-250 nm and IR radiation at the range of 1300-1400 cm⁻¹.

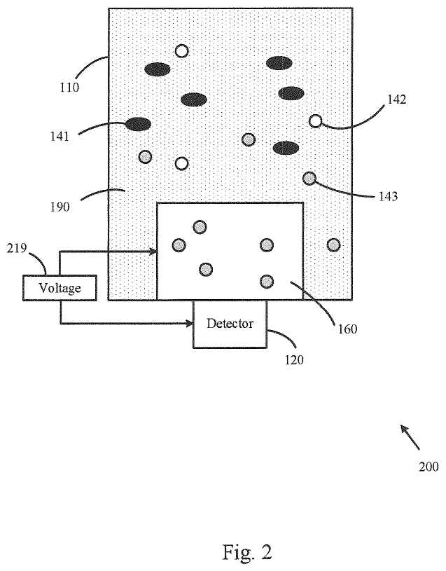

FIG. 2 illustrates electrical instrument 200 which integrates sorption material 160 using an electric signal (e.g., impedance, resistance, current, voltage, etc.) for sensing an analyte according to an embodiment of the disclosure. Electrical instrument 200 includes reservoir 110 that contains a liquid, such as water 190. Water 190 contains a plurality of analytes, including analytes 141 (black ovals), analytes 142 (white circles), and preferred analytes 143 (gray circles). Electrical instrument 200 includes, for example, voltage source 219 and detector 120 that traps, and pre-concentrates preferred analyte 143 in sorption material 160.

This category of electrical instrument 200 includes ChemFETs, electrochemical, resistive and similar measurement devices. The reference electrodes (for electrochemical), source and drain (for ChemFET) and other details are omitted for brevity. The essential point is the integration of sorption material 160 that can enhance the electrical signal due to trapping of the analyte 143 of interest. Trapped analyte 143 may change the resistivity of sorption material 160. Analyte 143 may affect the passage of electrons through sorption material 160 and vary the measured voltage, current, impedance and other properties. Therefore, the electronic measurements may change proportionally to the amount of the preferentially trapped analytes 143. In accordance with the disclosed method, these time-resolved electrical signals are continuously analyzed using kinetic models that fit the sorption behavior as data is collected, enabling prediction of the equilibrium analyte concentration prior to actual equilibrium, and thereby improving the speed and accuracy of detection.

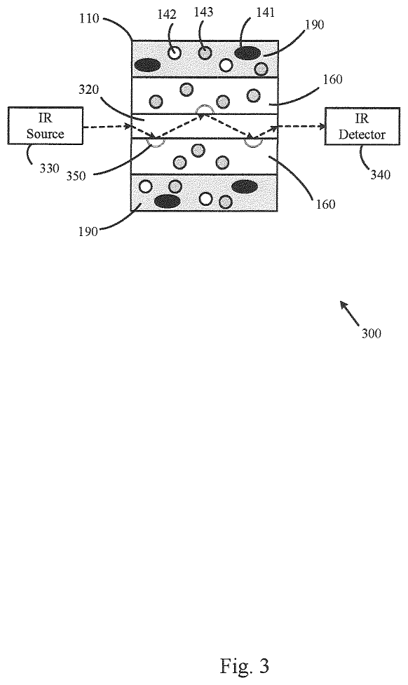

FIG. 3 illustrates optical instrument 300 which integrates sorption material 160 using infrared spectral range radiation according to an embodiment of the disclosure.

Optical instrument 300 includes reservoir 110 that contains a liquid, such as water 190. Water 190 contains a plurality of analytes, including analytes 141 (black ovals), analytes 142 (white circles), and preferred analytes 143 (gray circles). Optical instrument 300 includes, for example, waveguide 320, infrared (IR) source 330, and IR detector 340 that traps, and pre-concentrates preferred analyte 143 in sorption material 160.

For infrared radiation (1-14 μm), the radiation may be strongly absorbed by water 190 (or other liquids). To overcome this challenge and ensure that radiation reaches IR detector 340, an attenuated total reflection (ATR) method may be used. In this method, the radiation passes through waveguide 320 rather than directly through water 190 and the sensing of the contaminants proceeds using evanescent field 350. In this case, sorption material 160 may be coated or deposited or attached to the surface of waveguide 320 to trap analytes 143 from the surrounding liquid 190 in a close proximity to waveguide 320 within the reach of evanescent field 350.

Sorption materials—The sorption materials 160 for capturing contaminants from water and other fluids may include the following groups: i) zeolites; ii) granular activated carbon and activated carbon fibers; iii) ion-selective resins and polymeric-based sorption materials in general; iv) metal-organic frameworks (MOFs); v) nanoparticles and nanocomposites; vi) materials from natural plants, such as coconut carbon from coconut shell, peat moss, rice husk; vii) beads, pellets or granules of sodium or potassium permanganate (e.g., KMnO₄); viii) impregnated carbon; ix) metal oxides (ZnO or Al2O3); or x) molecular imprinted polymers (MIPs), as well as mixtures/composites of the above materials.

Some materials are more selective than others to certain analytes, while others lack selectivity. The present disclosure describes the integration of these sorption materials into measurement tools as in FIGS. 1-3 above for signal enhancement and methods for subsequent analysis of the enhanced signals. The signal enhancement is achieved by increasing the number of analytes 141-143 in the sorption material 160. The trapping may be selective or non-selective, depending on the application and the instrument in which the sorption material 160 is integrated. Multi-analyte sensing can be effectively implemented, especially when spectroscopic methods allow differentiation of optical signatures corresponding to distinct trapped analytes. This approach was implemented by Max-IR Labs, Incorporated to detect PFAS, sulfates, and nitrates in reverse osmosis (RO) brine solutions using a single time-dependent kinetic measurement, where spectroscopic signal differentiation enabled successful multi-analyte analysis.

Descriptions of some sorption materials 160 are provided below:

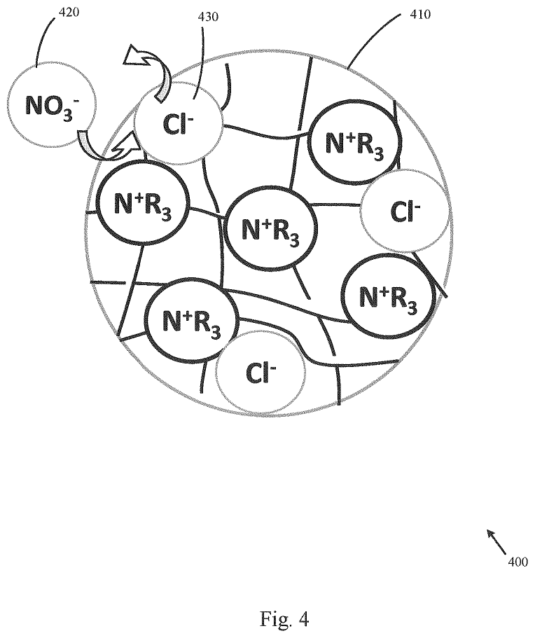

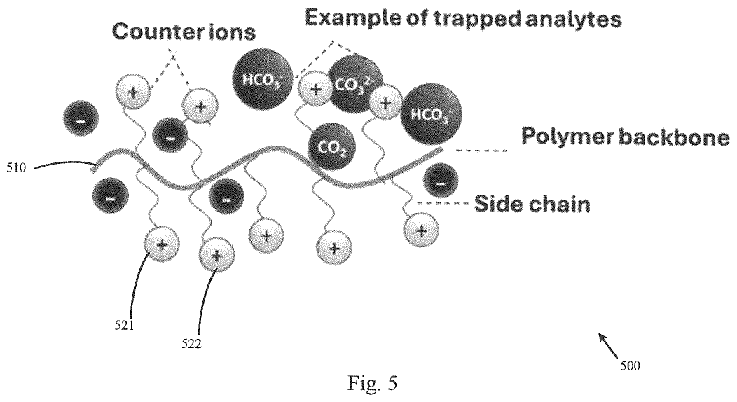

Polymeric materials—Polymeric materials include multiple materials, including ion-exchange resins and ion-exchanged ionomers as shown in FIG. 4 and FIG. 5.

FIG. 4 illustrates an ion exchange resin 400 according to an embodiment of the disclosure. FIG. 4 depicts counter-ion 430 from resin 400 and contaminant 420 from water. Cation and anion exchange resins are polymeric materials designed to remove specific ions from water 190 through an ion exchange process. These resins have a porous, cross-linked polymeric structure, typically made of polystyrene, which provides a stable framework as shown in FIG. 4. The ionic structure of the resins consists of fixed ion-exchange sites. Cation exchange resins have negatively charged sites that attract and hold positively charged counter-ions like sodium (Na⁺). Anion exchange resins may have positively charged sites that attract and hold negatively charged counter-ions like hydroxide (OH⁻). When water containing contaminants passes through these resins, the counter-ions on the resin are exchanged with contaminant ions in the water. For example, in a cation exchange resin, calcium ions (Ca²⁺) in the water replace sodium ions in the resin, while in an anion exchange resin, nitrate ions (NO₃⁻) in the water replace hydroxide ions in the resin. This process effectively traps the contaminants within the resin, resulting in purified water.

In context of this disclosure, these materials are utilized for trapping and preconcentrating contaminants from water. The trapped contaminants are then analyzed using various methods, such as described in FIGS. 1-3. Implementation of ion-exchange materials for measurements of nitrogen contaminants such as nitrate and ammonia in water and soils were described previously in prior Max-IR, Incorporated patents incorporated by reference above.

FIG. 5 illustrates an ion-exchanged ionomer 500 according to an embodiment of the disclosure. Ion-exchanged ionomers are known for their use in fuel cells. Ion-exchanged ionomers are specialized polymers that facilitate ion transport within a fuel cell, playing a crucial role in the electrochemical processes of the cell. Ionomer 500 includes a backbone structure 510 typically made of (but not limited to) polytetrafluoroethylene (PTFE) for chemical stability, with side chains 521 and 522 that may contain ion-exchange functional groups, such as sulfonic acid (—SO₃H) for proton exchange membranes (PEMs) or quaternary ammonium groups (—NR₃⁺) for anion exchange membranes (AEMs). These functional groups enable the selective transport of protons (H⁺) or hydroxide ions (OH⁻), essential for maintaining the cell's ion balance and facilitating the electrochemical reactions. The ion-exchange functional groups in the ionomers, such as sulfonic acid groups in PEMs or quaternary ammonium groups in AEMs, can interact with various analytes of interest in liquids. These polymers are excellent for integration with instruments, such as in FIG. 1. Integrated polymers act for capture and trapping of analytes for subsequent measurement using these analytical techniques. For example, Integrated polymers can be used from preconcentration of inorganic carbon in fluids including CO₂, bicarbonate and carbonate ions, enabling monitoring in various fluids.

Activated carbon—Materials from natural plants, such as peanuts and coconut shells, may be transformed into activated carbon through a process of pyrolysis followed by activation. During pyrolysis, these organic materials are heated in the absence of oxygen, decomposing them into a carbon-rich char. The char is then subjected to activation, which can be chemical or physical. Chemical activation involves treating the char with activating agents, like phosphoric acid or potassium hydroxide, while physical activation involves heating the char in the presence of activating gases like steam or carbon dioxide.

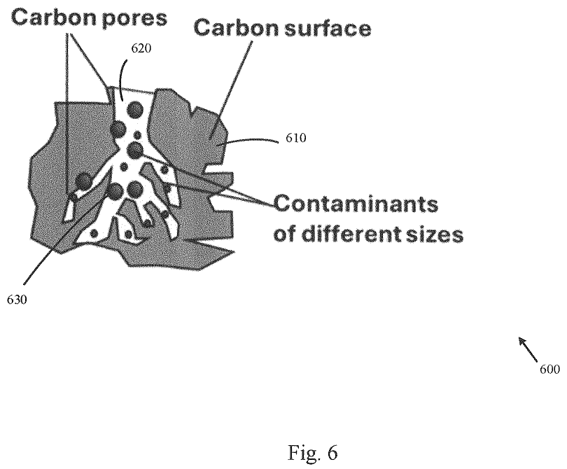

FIG. 6 illustrates activated carbon 600 according to an embodiment of the disclosure. The activated carbon 600 includes carbon surface 610, carbon pores 620 and contaminants 630 of different sizes. The activation process creates a highly porous activated carbon 600 structure with a large surface area, ideal for adsorbing contaminants 630. Activated carbon 600 made from peanut shells and coconut shells may effectively capture various contaminants from water, including organic compounds, heavy metals, and chlorine, by trapping them within its pores through adsorption. This makes activated carbon an efficient and sustainable material for water purification.

The activated carbon 600 derived from natural materials such as peanut shells, coconut shells, wood, and agricultural residues may be processed into granular activated carbon (GAC). Granular activated carbon refers to activated carbon that has been processed into small, granule-sized particles. The main difference lies in the form and specific application rather than the source material. GAC typically has larger particle sizes compared to powdered activated carbon (PAC) and may be used in various applications, such as water and air purification, where it can be employed in fixed-bed filters, fluidized beds, or as a filter medium. The properties of GAC, including pore size distribution, surface area, and adsorption capacity, can vary depending on the source material and the activation process used. For example, coconut shell-based GAC is known for its high hardness and extensive micropore structure, making it particularly effective for removing small organic molecules and impurities from water.

Other materials for activated carbon 600 may include other nut shells such as walnut, almond, and pecan shells are also used for their high lignin and cellulose content. Different types of wood, such as oak, pine, and bamboo, are commonly used for producing activated carbon due to their high carbon content and ease of activation. Materials like olive pits, apricot pits, and date seeds can be converted into activated carbon, taking advantage of their dense structure. Agricultural byproducts such as rice husks, corn cobs, and sugarcane bagasse are abundant and cost-effective sources for activated carbon production. Bone char, derived from animal bones, is a type of activated carbon used primarily for decolorizing and purifying sugar and water. Peat, an accumulation of partially decayed vegetation or organic matter, can be processed into activated carbon.

Zeolites—Zeolites absorb contaminants from water through a combination of ion exchange, adsorption, and molecular sieving. These naturally occurring or synthetic crystalline aluminosilicates have a porous structure with a high surface area and negatively charged frameworks, which attract and hold positively charged ions. When contaminated water passes through a zeolite, harmful cations like heavy metals (lead, cadmium, zinc) are exchanged with benign cations (sodium, potassium) present in the zeolite. Additionally, zeolites can adsorb organic molecules and some microorganisms into their pores. The specific size and shape of the pores allow zeolites to selectively trap certain contaminants while allowing clean water to pass through.

Integration of zeolites in instruments such as those in FIGS. 1-3 is proposed for trapping of contaminants from fluids, and subsequent evaluation of the contaminants trapped inside using analytical tools.

Metal-organic frameworks—Metal-organic frameworks (MOFs) trap contaminants from fluids through their highly porous and crystalline structure composed of metal ions or clusters connected by organic ligands. These MOFs may have large surface areas and tunable pore sizes, allowing the MOFs to selectively adsorb various contaminants. For instance, in the case of per- and polyfluoroalkyl substances (PFAS), which are persistent organic pollutants known for resistance to degradation, MOFs have been shown to effectively trap these compounds due to their ability to interact with the fluorinated chains through van der Waals interactions and hydrogen bonding within the pores. The mechanism involves PFAS molecules diffusing into the pores of MOFs where the PFAS molecules are held by weak interactions with the MOFs surface.

Integration of MOFs in analytical tools such as those in FIGS. 1-3 increases the selective trapping capability. Combined with the structural versatility and high adsorption capacities of MOFs, the MOFs are ideal for integration into measurement instruments for trapping of contaminants, thus enhancing the detection of the instruments.

Functionalized and impregnated materials—Impregnation involves treating various materials, such as carbon, zeolites, porous alumina, silica and others, with a chemical solution to modify their surface properties, enhancing their suitability for specific applications, such as gas adsorption in air purification or catalysis. The present disclosure focuses on using these properties for trapping contaminants from fluids.

For example, the process of impregnating host materials (such as carbon powders, or alumina or silicon pellets) involves treating the materials with chemicals to enhance the ability to adsorb specific gases from the air. For removing basic pH gases (8-14 pH), such as ammonia and amines, these materials are impregnated with acidic compounds like sulfuric acid or phosphoric acid. This impregnation modifies the surface chemistry of the carbon, enabling the carbon to effectively capture basic gases through acid-base reactions. Alternatively, for removing acidic gases (1-7 pH) like hydrogen sulfide and sulfur dioxide, carbon materials are impregnated with alkaline compounds such as potassium hydroxide or sodium carbonate. These alkaline impregnates neutralize acidic gases upon contact, converting the gases into non-volatile salts or water-soluble compounds that adhere to the carbon surface. Here, these impregnated materials are adopted for capturing contaminants from fluids and subsequent detection through integration with measurement systems.

By way of example, permanganate-impregnated alumina pellets or beads remove methanol from water through a chemical oxidation process. The permanganate acts as a strong oxidizing agent, reacting with methanol to convert methanol into carbon dioxide and water, thereby eliminating the contaminant. The alumina substrate provides a high surface area for the permanganate, enhancing the contact and reaction efficiency with methanol molecules. Other materials suitable for impregnation are carbon, silica gel, zeolites, and even certain polymers. Each material offers unique characteristics such as surface area, pore size distribution, and chemical reactivity, influencing the efficiency and application of permanganate impregnation for water treatment purposes.

Integration of this type of material in the analytical instrument may result in accumulation of intermediate products (in addition or instead of primary analytes). By way of example, in the process of methanol oxidation using permanganate impregnated materials, intermediate oxidation products, such as formaldehyde, formic acid, and potentially other compounds may adsorb or accumulate within the impregnated materials. These intermediate products may interact with the surface of the host material, depending on its properties such as surface chemistry and pore structure.

Methanol may also adsorb onto the impregnated material surface, especially if the material has a high surface area and specific affinity for alcohol molecules. This adsorption can affect the overall capacity of the impregnated material to oxidize and remove methanol from the solution or air. Thus, impregnated materials integrated in analytical tools may analyze the directly trapped materials (such as methanol) or the reaction product (in this case formaldehyde, formic acid, and potentially other compounds).

Coatings and stand-alone sorbent materials for integration with instruments—Integration with analytical instruments depends on the type of instrument. For example, for optical instruments, the integration can be by coating of the optical plates, transparent in the spectral range of interest, by the sorption materials. Alternatively, the materials can be used as stand-alone membranes; or incorporated into polymeric meshes that allows both the capture of the contaminants and passing of the beam through these materials. When reflection mode of operation is chosen, the sorption materials can be deposited or adhered to reflective plates (gold-coated plates, etc.), enabling interaction of the probing beam with the analytes trapped in the sorption material.

Similarly, instruments that rely on electric mode of detection utilize coating of electrodes for trapping of the contaminants; or plates/surfaces that are utilized for validation of change in resistance, impedance, or related measured values. Coating of optical elements (waveguides, or support surfaces such as windows, or optical elements in the path of the probing beam) for trap-and-measure can vary in material structure and method of coating application.

Coated waveguides designed for operation in the infrared spectral range may be implemented in the optical instrument in FIG. 3. The coatings may be performed on silver halide waveguides, which is one example of material that is transparent in infrared spectral range. The key considerations for choosing the materials for optical elements, such as waveguides and optical plates, are (a) sufficient transmission and the relevant spectral range; and (b) sufficient stability in water. The latter may be less important if the coating provides a good degree of protection against the fluid in which it is submersed. For infrared spectral range, optical elements can be chalcogenides, fluoride glasses, silver halides, germanium, InP, GaAs, GaSb, KBr, KRS-5, ZnSe, Zinc sulfide, AMTIR, Silicon, etc. For the visible or UV spectral ranges, examples include quartz, sapphire, fused silica, fluorides (e. g. calcium fluoride, lithium fluoride), and many others.

Coating of infrared waveguides with sorption materials, such as in FIG. 3, presents examples of: i) coating using activated carbon, ii) coating using ion-exchange resin; and iii) direct coating of a polymer on waveguides using a film casting (FC) method. In case of resin, the resin particles may be milled first to reduce size to fine particle size to enable better packing and homogeneity, as well as a better control over the layer thickness. Other sorption materials may undergo a similar pretreatment to reduce size, or activate for better attachment to the surface, or a more efficient trapping. The deposition of activated carbon and resin in a process may consist of several steps. First, the waveguides may be treated with H₂O₂ and acid to activate the surface with hydroxyl groups. Other method of surface pre-treatment can be applied, for activation with other chemical or atomic groups, or this step can be omitted if the surface is sufficiently reactive. This step may be followed by activation using (3-Aminopropyl) triethoxysilane (APTES). APTES bonds to the surface hydroxyl groups and acts as an adhesion layer. Other molecules can be used for adhesion, for example, amino-containing surface or others. Milling of activated carbon or ion-exchange resins is a step that reduces the dimension of these particles and create homogeneous thin layers. The activated or pre-treated optical elements are then coated by the material of interest.

When working in the IR spectral range using ATR configuration, the penetration depth of the evanescent field generated by total internal reflection depends on the refractive indices of the waveguide, the surrounding medium (including any coating), and the wavelength of the radiation (FIG. 3). If the detection of the water/coating interface is required, the coated layer thickness and refractive index should be matching the penetration depth of the IR radiation in the ATR configuration. Thinner coatings allow for faster analyte diffusion to the waveguide surface, enabling quicker sensing responses. However, they offer limited capacity for analyte accumulation and may require regeneration more quickly during prolonged measurements. In contrast, thicker coatings can accommodate a greater number of analyte molecules, improving sensitivity and measurement stability over time. They also serve to spatially separate the waveguide surface from interfering species or debris at the water interface, which may otherwise contribute to unwanted background signal. The trade-off is that analytes must diffuse a longer distance to reach the sensing region within the evanescent field, potentially increasing the response time.

When working with optical elements in the way of the beam (FIG. 1), the thinner layers are beneficial for reducing absorption and scattering effects of the radiation. However, if the coating material is sufficiently transparent in the spectral range of interest, the coating thickness can be optimized to accommodate higher number of the absorption sites for analytes of interest, while keeping the intensity of the probing radiation sufficient to prevent reduction in the signal-to-noise ratio of the measurement.

Trap-and-measure process—Examples of integration of the sorption material in the measurement apparatus may include an IR-ATR type of measurement, but the trapping and data analysis processes are similar, and often identical across different types of instruments. The primary distinction lies in the measurement method. In this example, the sorption material may be integrated into the instrument by coating on infrared fibers (as in FIG. 3). The fibers may be coated using nanosized-particle resins (hereafter, referred as “NP”) as well as film-casted polymers (hereafter referred as “FC”). The following analytes may be present as contaminants in the analyzed fluid:

per- and polyfluoroalkyl substances (PFAS)—PFAS (per- and polyfluoroalkyl substances) are a group of synthetic chemicals used in numerous industrial and consumer products for the water and grease-resistant properties. These substances are characterized by strong carbon-fluorine bonds, making them highly persistent in the environment and resistant to degradation. PFAS contaminants have been linked to health concerns due to their bio accumulative nature and widespread presence in water sources, leading to regulatory scrutiny and remediation efforts worldwide.

Nitrate—Measurement of this contaminant in water is crucial due to its potential health impacts, particularly in high concentrations. Elevated nitrate levels can indicate contamination from agricultural runoff or wastewater, posing risks such as methemoglobinemia (“blue baby syndrome”) in infants and potential health issues in adults. Monitoring nitrate levels helps ensure water safety and informs necessary remediation actions to protect public health and the environment.

### Trap and Measure of PFAS Contaminants

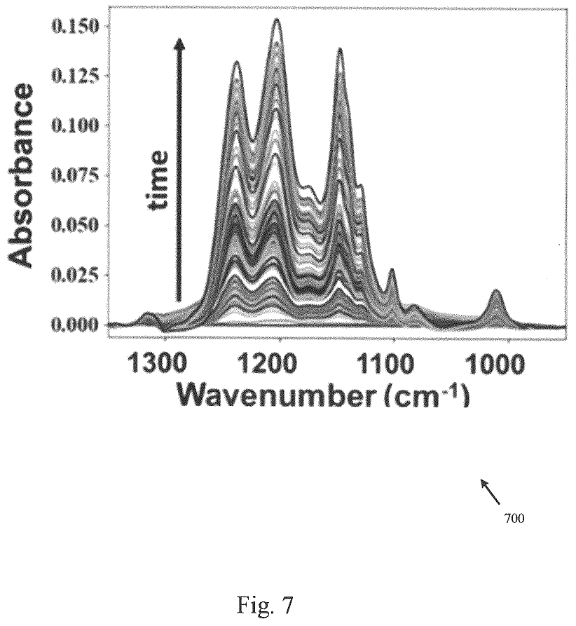

FIGS. 7-10 illustrate raw IR spectra, collected during accumulation of PFAS in the FC coating. Experimental conditions in an experiment may include water with 25 ppm PFOA pumped into a 25 mL cell that contains the waveguides (similar to FIG. 3). The flow is stopped, and the process of PFAS trapping in the coating may be monitored using the infrared FTIR spectrometer integrated with the sorbent material. The spectra in FIGS. 7-10 show a typical fingerprint signature due to the stretching carbon-fluorine (C—F) vibrations of PFOA. The intensity of the absorption bands increases as a function of time due to sorption of PFAS in the coated waveguide surface.

FIG. 7 is a graph 700 illustrating an IR peak area of the C—F bond showing development as a function of time, during PFAS trapping in FC waveguide coating according to an embodiment of the disclosure.

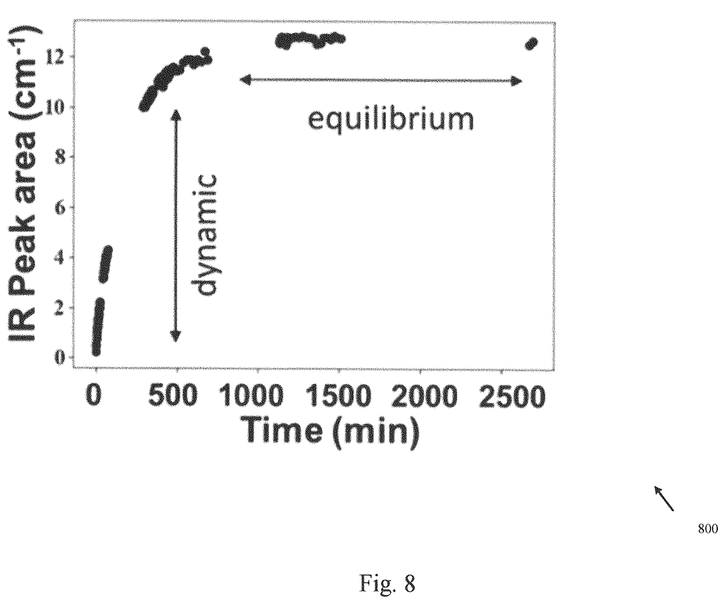

FIG. 8 is a graph 800 illustrating calculated IR peak area of PFAS as a function of time in an FC coated waveguide according to an embodiment of the disclosure.

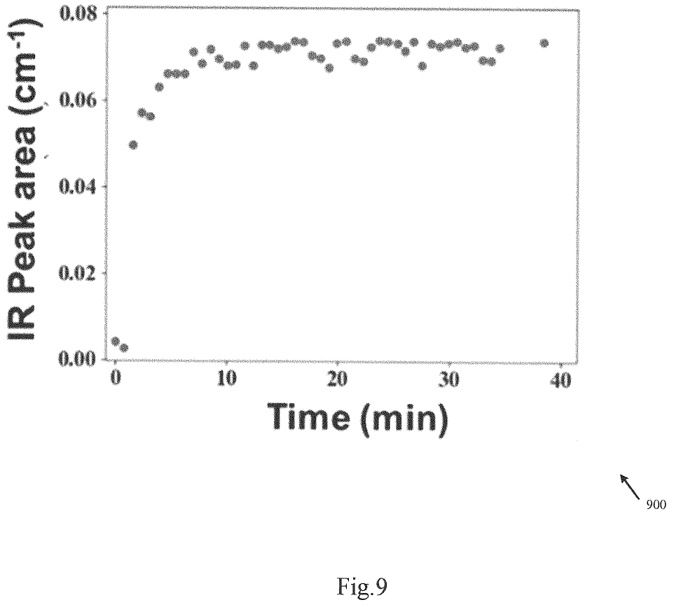

FIG. 9 is a graph 900 illustrating calculated IR peak area of PFAS as a function of time in an NP coated waveguide according to an embodiment of the disclosure.

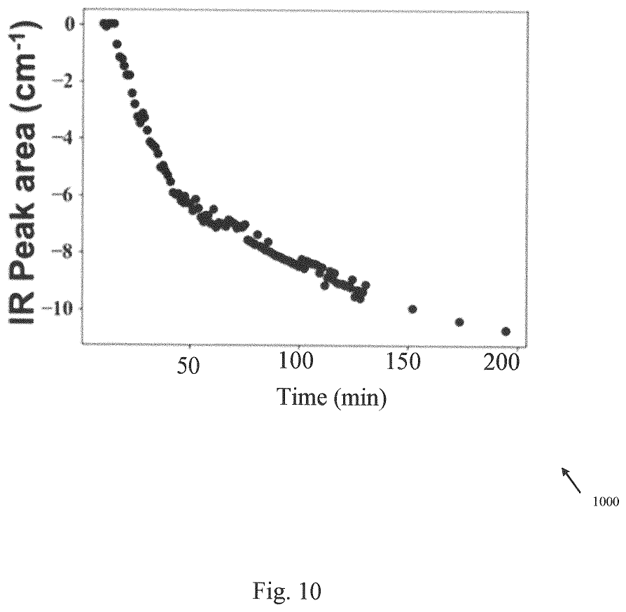

FIG. 10 is a graph 1000 illustrating IR peak area during monitoring of FC coating regeneration salt solution according to an embodiment of the disclosure.

FIG. 8 shows the integrated area of the IR absorption bands as a function of time for FC waveguides. For comparison, FIG. 10 shows kinetics of PFAS trapping on a Nanoparticle (NP)-coated waveguide. Data analysis and fits indicate a 40× IR signal enhancement in NP-coated fibers vs. uncoated (bare) fibers. The FC coating resulted in 8,000× enhancement relative to a bare fiber (without the integrated sorbent material). The superior enhancement of the FC coating is due to the higher density of the polymeric network in the coating compared to that of the NP coating of loosely bound particles. This allows for more PFAS binding sites within the sensing region near the waveguide surface. Additionally, the roughness of the NP coating reduces the effective path length of the evanescent field within the coating, resulting in weaker IR absorption bands. This can be further optimized by enabling more surface area of sorbent materials interact with the analytes of interest (PFAS in this case).

IR data from NP-coated waveguides (FIG. 10) indicates achievement of steady-state within 8 minutes of PFAS introduction. For FC-coated waveguides, the steady-state is achieved within 500 minutes. The reason is the difference in the coating thickness: The NP coating was roughly 2 μm thickness, while the FC coating was 30 μm thick. For comparison, the infrared evanescent field penetration is at the order of 2 μm into the coating. In the case of NP, only a single layer is deposited on the waveguide surfaces through direct attachment of NP on APTES. PFAS that is trapped on the surface of the NP-coating is thus immediately detected by the evanescent field.

Alternatively, in a 30 μm thick FC film, the transport toward the evanescent field is diffusion limited. Since the chemical structure of the NP-coating and the FC-coating is essentially identical, similar dynamics are expected upon thickness reduction. Optimizing the thickness of the FC coating (for example, by tuning the concentration of the polymer in the casting solution and deposition duration), can lead to further signal enhancement due to the enlarged capacity for trapping of contaminants and bringing the trapped contaminants close to the probing radiation.

Regeneration—Real-time monitoring of the FC coating regeneration in salt solution is demonstrated in FIG. 9. Similar to PFAS sorption, regeneration kinetics are also governed by the thickness of the FC coating. The regeneration performance can be tuned by using appropriate conditions (for example, concentration of the regenerating solution; pH, temperature, etc.).

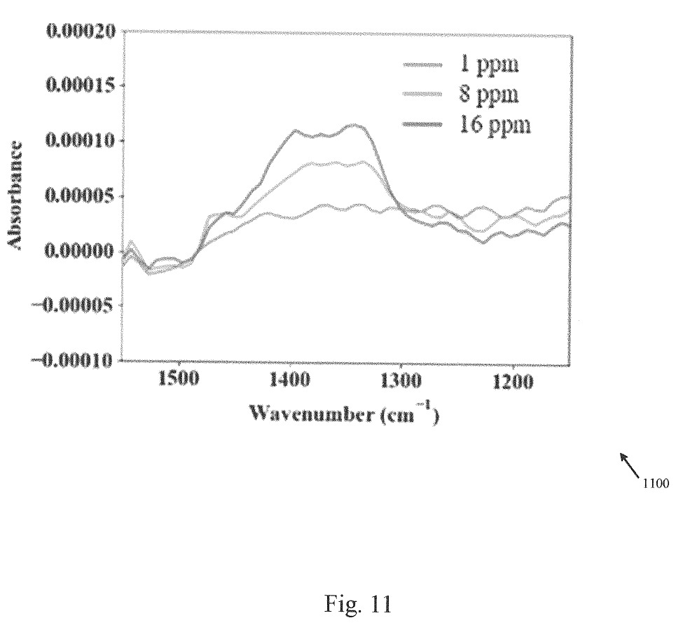

FIG. 11 is a graph 1100 illustrating IR absorption peaks of 1 ppm, 8 ppm, and 16 ppm nitrate solutions measured using an uncoated fiber according to an embodiment of the disclosure. The resulting IR absorbance peak areas are 1.4*10⁻³ cm⁻¹, 4.6*10⁻³ cm⁻¹, and 8.5*10⁻³ cm⁻¹, respectively.

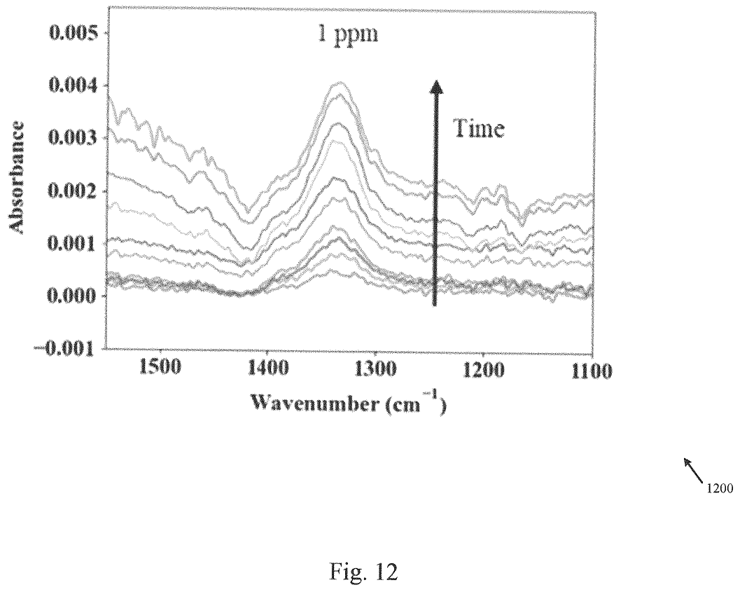

FIG. 12 is a graph 1200 illustrating IR absorption peaks of 1 ppm nitrate solution measured using a coated fiber according to an embodiment of the disclosure. The peak area increases from initial measurement (bottom) to final measurement (top) over the course of 100 minutes. Every fifth measurement is shown. Measurements were taken approximately every 2 minutes over a period of 100 minutes. The first measurement was taken 2 minutes after the introduction of the nitrate solution and is shown at the bottom with the smallest nitrate absorption peak. The final measurement was taken 100 minutes after the introduction of the nitrate solution and is shown at the top with the largest nitrate absorption peak.

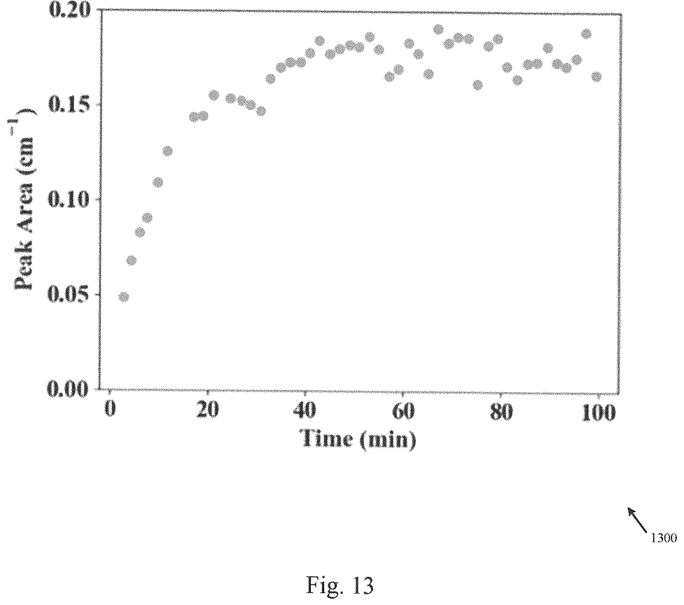

FIG. 13 is a graph 1300 illustrating nitrate IR absorption peak area from measurements shown in FIG. 12 plotted as a function of time according to an embodiment of the disclosure. The enhanced peak area is due to adsorption of nitrate from the solution into the fiber coating. The evanescent field of the IR radiation travelling through the fiber extends approximately 3 μm beyond the surface of the fiber. As nitrate is adsorbed into the coating within this sensing region, the volume density of nitrate increases compared to that in the solution, enhancing the IR absorption signal.

As shown in the peak area vs. time plot (FIG. 13), the initial peak area of 0.05 cm⁻¹ measured 2 minutes after introduction of the 1 ppm nitrate solution to the sensor cell with coated fiber increases to 0.18 cm⁻¹ after about 50 minutes and remains at this saturated level for the remainder of the 100 minute experiment. The total peak area increase per ppm of nitrate after saturation is reached is >300× higher compared to the uncoated fiber.

Data analysis—The rate of adsorption of nitrate into the coating can be modelled by a pseudo-second-order kinetic expression:

$${dq}_{t} / dt = {{k}_{2} ( {q}_{t} - {q}_{e} )}^{2} \qquad ( Equation 1 )$$

where:
qₜ is the concentration of nitrate in the fiber at time t,
k₂ is the pseudo-second-order rate constant, and
qₑ is the equilibrium concentration of nitrate in the fiber.

The kinetic model in Equation 1 assumes that the rate of change in the concentration of nitrate in the fiber increases with time proportionally to the square of the difference between the concentration in the fiber at time=t and the equilibrium concentration in the fiber. The kinetic curve transitions from rising to leveling off (“plateau region”), signaling that the equilibrium is achieved. Under the assumption that the equilibrium concentration of nitrate in the fiber is proportional to the concentration of nitrate in the solution, then by the Beer-Lambert law the IR absorption peak area at the “plateau” region (when the kinetic curve levels off—we refer to this point as “saturation”) will also be proportional to the nitrate concentration in the solution. Thus, the nitrate concentration in the solution can be determined by applying a linear standard calibration curve to the saturation peak area. Here, saturated IR absorption peak area (when the plateau in the kinetic curve is achieved) may be used in place of nitrate concentration.

Example analysis of Nitrate data—The time to measure the saturated IR absorption peak area, and thus nitrate concentration, in this case is approximately 50 minutes. To reduce the time of the nitrate concentration measurement, the pseudo-second-order model may be used to predict the saturated peak area beginning after the first two measurements. After applying the boundary condition q₀=0 the differential Equation 1 can be solved and rearranged into the linearized form:

$$t / {q}_{t} = 1 / {q}_{e}^{2} {k}_{2} + ( 1 / {q}_{e} ) t \qquad ( Equation 2 )$$

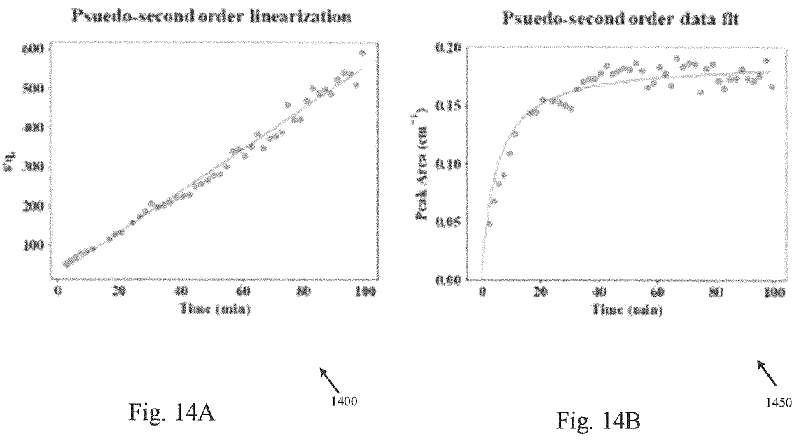

FIG. 14A is a graph 1400 that is data linearized according to a pseudo-second-order kinetic model with a fit line according to an embodiment of the disclosure.

FIG. 14B is a graph 1450 that shows the original data shown on peak area vs. time axes with pseudo-second order fit line from FIG. 14A according to an embodiment of the disclosure.

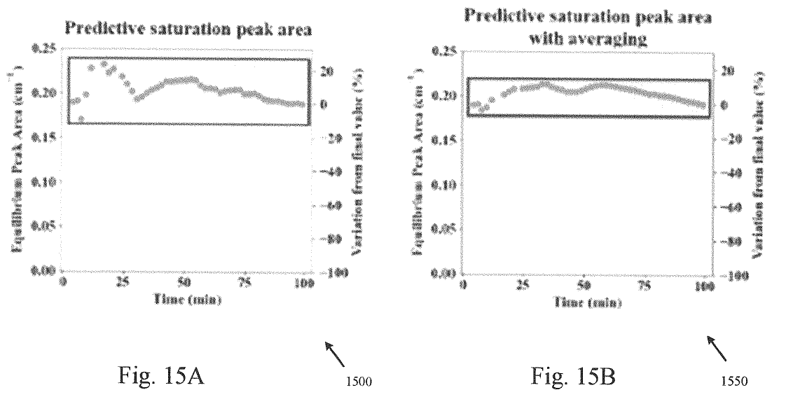

FIG. 15A is a graph 1500 that shows the predictive equilibrium peak area (left y-axis) vs. time with percent variation from the final value (right y-axis) according to an embodiment of the disclosure.

FIG. 15B is a graph 1550 that shows the predictive peak area vs. time as in FIG. 15A with a 10 data point moving average window applied according to an embodiment of the disclosure.

In FIG. 14A, the data from FIG. 13 is plotted on the modified y-axis t/qₜ as shown in Equation 2. The linear fit to the data is shown as the orange line with slope

$$\frac{1}{{q}_{e}}$$

and intercept

$$\frac{1}{{q}_{e}^{2} {k}_{2}} .$$

FIG. 14B shows the data on the original axis along with the model fit from FIG. 14A. To estimate the equilibrium peak area at any point in time t, the fit to the linearized Equation 2 may be applied only to the data collected up to time t with the equilibrium peak area taken as the inverse of the slope of the fit line.

The result of this procedure for each time, t, is shown in FIG. 15A as a plot of the predicted equilibrium peak area vs. time. The right y-axis shows the percentage deviation from the final measured equilibrium peak area at each point in time, and the shaded box shows the maximum extent of the deviation, which is within ±25% within the first 4 minutes of measurement.

In FIG. 15B, a moving average based on the most recent 10 data points (if available) is applied to smooth the output, resulting in a maximum deviation from the final value of just ±12% beginning with data available within 4 minutes of the start of measurement. By incorporating the above procedure into the sensor control software real-time estimates of the equilibrium concentration are available from the sensor shortly after measurements begin.

### Analysis of PFAS Data—

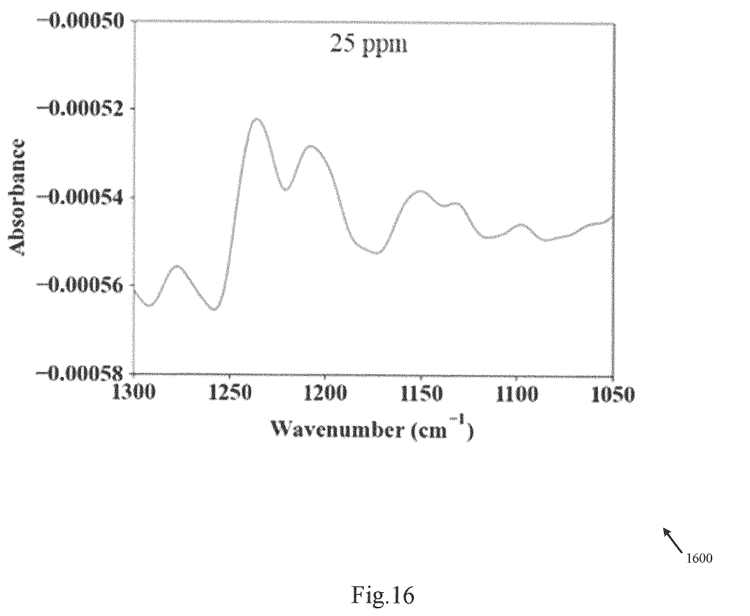

FIG. 16 is a graph 1600 that shows the IR absorption spectrum from the C—F bonds of a 25 ppm solution of PFOA in water measured using an uncoated fiber according to an embodiment of the disclosure.

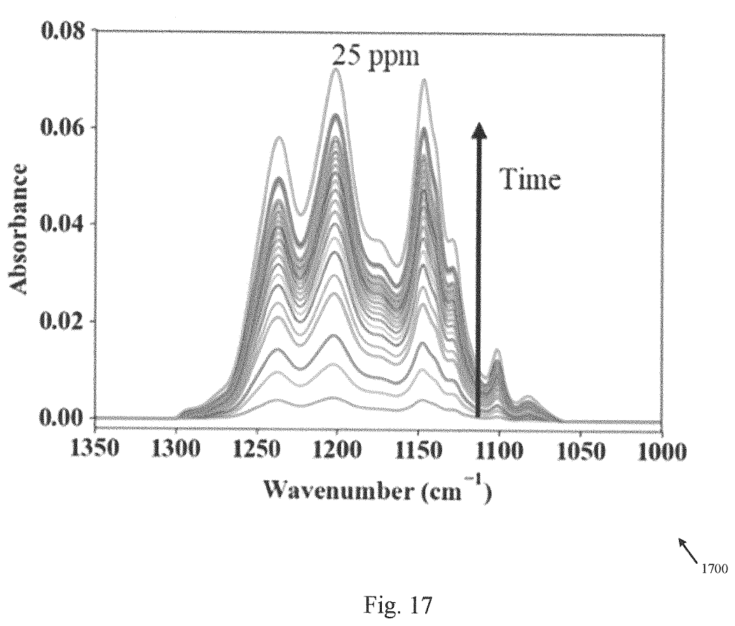

FIG. 17 is a graph 1700 that shows the IR absorption spectrum of a 25 ppm solution of PFOA in water measured using a coated fiber according to an embodiment of the disclosure.

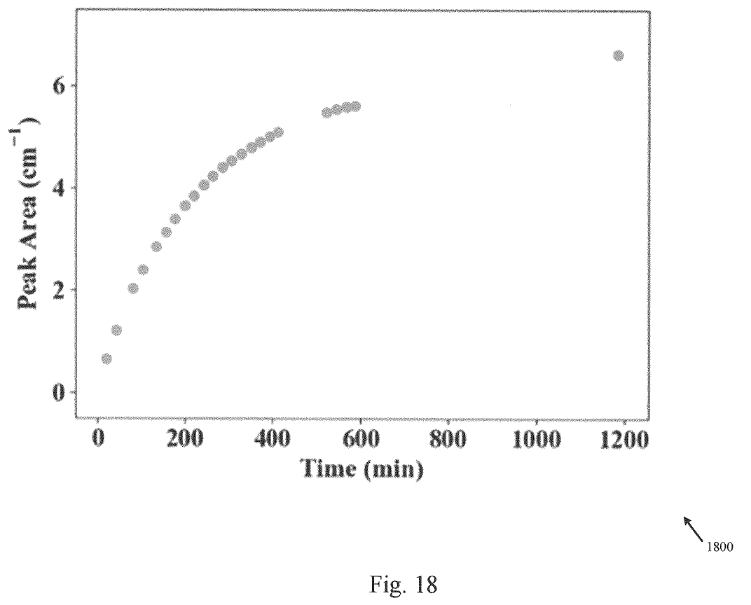

FIG. 18 is a graph 1800 that shows the PFOA IR absorption peak area from measurements shown in FIG. 17 plotted as a function of time according to an embodiment of the disclosure.

In FIG. 16, the absorbance peak height is approximately 2*10⁻⁵, just above the detection limit using an uncoated fiber. To decrease the detection limit, fibers may be coated in an ISM material which is selective to PFAS compounds, with the results shown in FIG. 17 and FIG. 18. In FIG. 17, the greatly enhanced signal from PFOA can be seen with an absorbance peak height of about 5*10⁻³ in the first measurement (bottom curve) after 20 minutes of exposure of the fiber to the 25 ppm PFOA solution. After 20 hours of exposure, the absorption peak height increases to 0.07 (top curve). The peak area vs. time for these measurements is shown in FIG. 18. The selectivity of ISM enables sensing of specific analytes, such as PFAS, in complex matrices, that contain additional contaminants, with examples of reverse osmosis (RO) brines, industrial wastewater, and other complex fluids.

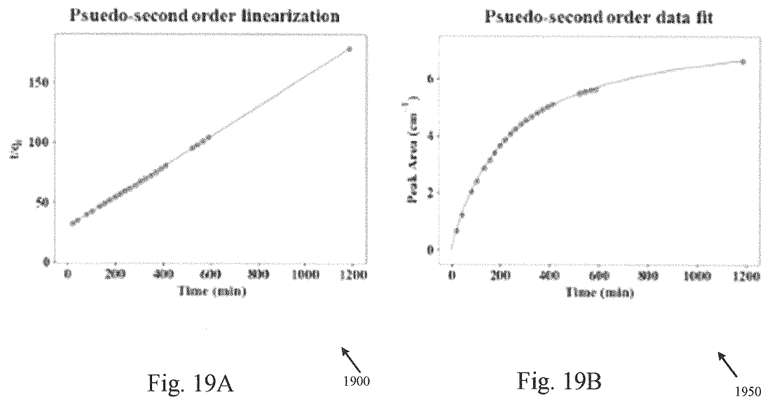

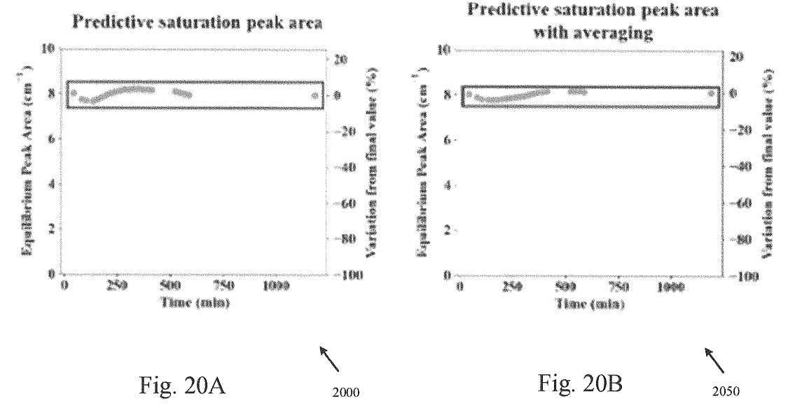

FIGS. 19A and 19B and FIGS. 20A and 20B illustrate PFOA kinetic model fitting.

FIG. 19A is a graph 1900 that shows data linearized according to a pseudo-second-order kinetic model (circles) with a fit line according to an embodiment of the disclosure.

FIG. 19B is a graph 1950 that shows the original data (circles) on peak area vs. time axes with pseudo-second-order fit line from FIG. 19a according to an embodiment of the disclosure.

FIG. 20A is a graph 2000 illustrating predictive equilibrium peak area (left y-axis) vs. time with percent variation from the final value (right y-axis) according to an embodiment of the disclosure.

FIG. 20B is a graph 2050 illustrating the predictive peak area vs. time as in FIG. 20A with a 10 data point moving average window applied according to an embodiment of the disclosure.

To decrease the measurement time from 20 hours to 40 minutes, the same kinetics fitting procedure as described above may be followed. FIG. 19A shows the linearized data from FIG. 18 (circles) and the fit line. In FIG. 19B, the fit line is displayed over the original data (circles). After applying the predictive peak area algorithm, the peak area at the “plateau” can be determined to within 4% of the final measured value after just two measurements (40 minutes), as shown in FIG. 20A. Again, a moving average is used on the most recent 10 data points (when available) as shown in FIG. 20A to reduce the variation in the data with time more quickly.

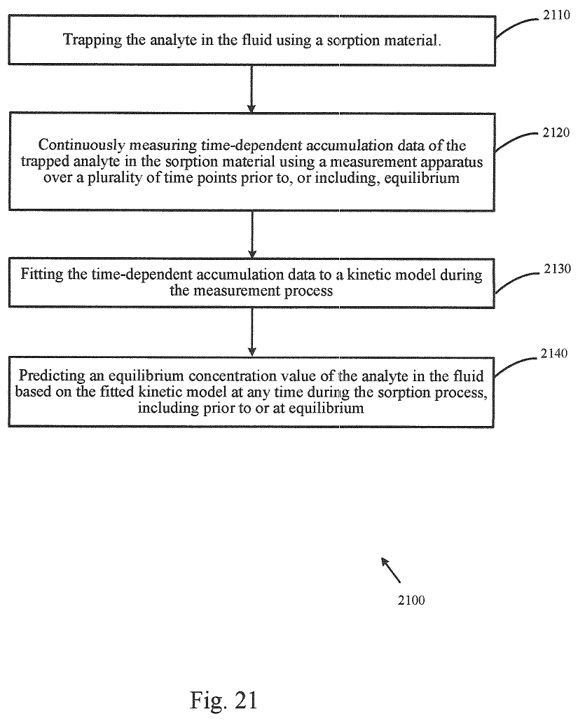

FIG. 21 illustrates a method for detection of an analyte in a fluid according to an embodiment of the disclosure. In 2110, the method traps the analyte in the fluid using a sorption material. In 2120, the method continuously measures time-dependent accumulation data of the trapped analyte in the sorption material using a measurement apparatus over a plurality of time points prior to, or including, equilibrium. In 2130, the method includes fitting the time-dependent accumulation data to a kinetic model during the measurement process. In 2140, the method includes predicting an equilibrium concentration value of the analyte in the fluid based on the fitted kinetic model at any time during the sorption process, including prior to or at equilibrium.

The present disclosure describes the capability to predict the concentration during the initial observations and/or initial onset of the measurements which allows data extrapolation and prediction of the equilibrium concentration values.

An alternative approach is to train AI to predict the behavior of trapping and measurement without the explicit application of the model in Equations 1 and 2, which are self-evident given the measured datasets that follow this trapping kinetic behavior. Moreover, AI-based training is more inclusive, as it can sub-categorize kinetics based on specific trapping/sorption materials, interactions with trapped analytes, and the surrounding interfering chemicals within the fluid matrix. Therefore, when properly trained, it can provide more precise concentration values even prior to reaching the equilibrium. The disclosed data analysis technique allows prediction of the concentration of the contaminants in the surrounding fluid at the initial stages of analyte trapping in the sorption material integrated within a measurement instrument.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A method for detection of an analyte in a fluid, the method comprising:
trapping the analyte in the fluid using a sorption material;
continuously measuring time-dependent accumulation data of the trapped analyte in the sorption material using a measurement apparatus over a plurality of time points prior to, or including, equilibrium;
fitting the time-dependent accumulation data to a kinetic model during the measurement process; and
predicting an equilibrium concentration value of the analyte in the fluid based on the fitted kinetic model at any time during the sorption process, including prior to or at equilibrium.

2. The method as set forth in claim 1, wherein the kinetic model describes a rate of analyte sorption and comprises one or more kinetic models selected from mechanistic, empirical, or rate-based models, including but not limited to:
Langmuir adsorption kinetics,
pseudo-first order,
pseudo-second order, intraparticle diffusion,
Elovich-type models, or
data-driven predictive models, including but not limited to:
regression, curve-fitting, or machine-learning-based models trained to infer equilibrium behavior based on sequential measurements; and
wherein the kinetic model comprises a pseudo-second-order kinetic expression:
$$d {q}_{t} / dt = {{k}_{2} ( {q}_{t} - {q}_{e} )}^{2} ,$$
where:
qₜ is the amount of analyte in the sorption material at time t,
k₂ is the pseudo-second-order rate constant, and
qₑ is the equilibrium concentration value of the analyte in the sorption material.

3. The method as set forth in claim 2, wherein the measuring apparatus applies the kinetic model to the time-dependent accumulation data in real time during the measurement process to dynamically estimate the equilibrium concentration of the analyte in the sorption material and infer the concentration of the analyte in fluid.

4. The method as set forth in claim 1, wherein the measurement apparatus determines the time dependent accumulation data by analyzing a time-dependent signal.

5. The method as set forth in claim 4, wherein the time-dependent signal comprises an optical signal, including spectroscopic signals.

6. The method as set forth in claim 5, wherein the measurement apparatus predicts the equilibrium concentration value by fitting a kinetic profile of the time-dependent optical signal, including spectroscopic data, collected during the sorption process.

7. The method as set forth in claim 1, wherein the time-dependent signal comprises an electrical signal.

8. The method as set forth in claim 7, wherein the measurement apparatus predicts the equilibrium concentration value by fitting a kinetic profile of the time-dependent electrical signal collected during a process of trapping the analyte.

9. The method as set forth in claim 1, wherein the kinetic model fitting is dynamically updated in real time with each subsequent measurement point collected during the process of trapping the analyte, thereby continuously refining the predicted equilibrium concentration value of the analyte.

10. The method as set forth in claim 9, wherein the accuracy of the predicted equilibrium concentration value improves progressively as additional time-dependent measurement data points are collected and incorporated into the kinetic model fitting.

11. The method as set forth in claim 1 wherein the fluid comprises one of:
i) water;
ii) an organic or inorganic solvent; or
iii) a non-aqueous fluid.
